(19) United States
(12) Patent Application Publication
(10) Pub. No.: US 2007/0073184 A1
(43) Pub. Date: Mar. 29, 2007

(54) Sensor Device for Detecting LEEG Signals and Detecting Method Thereof

(75) Inventors: Chih-Cheng Lu, Taipei City (TW); Gin-Shin Chen, Changhua City, Changhua County (TW); Sheng-Fu Chen, Zhunan Town, Miaoli County (TW); Ming-Shaung Ju, Tainan City (TW); Chou-Ching Lin, Tainan City (TW)

Correspondence Address: AKIN GUMP STRAUSS HAUER & FELD L.L.P., ONE COMMERCE SQUARE, 2005 MARKET STREET, SUITE 2200, PHILADELPHIA, PA, 19103, US

(73) Assignee: NATIONAL HEALTH RESEARCH INSTITUTE, Zhunan (TW)

(21) Appl. No.: 11/534,807

(22) Filed: Sep. 25, 2006

Related U.S. Application Data
(60) Provisional application No. 60/720,436, filed on Sep. 26, 2005.

(51) Int. Cl. A61B 5/04

(52) U.S. Cl. 600/544

(57) ABSTRACT

A sensor device that detects Laplacian electroencephalogram (LEEG) signals includes a signal acquisition module placed on a scalp of a subject to acquire brain signals. A signal processor is coupled or connected to the signal acquisition module to perform a Laplacian operation on the signals acquired by the signal acquisition module such that the noise signal is reduced to yield an analog LEEG signal with a high signal-to-noise (S/N) ratio.

### CROSS REFERENCE TO RELATED APPLICATION

This application claims priority to U.S. Provisional Patent Application No. 60/720,436 filed on Sep. 26, 2005 entitled “Sensor Device for Detecting LEEG Signals and Detecting Method Thereof.”

### BACKGROUND OF THE INVENTION

The present invention relates to the field of electrophysiology, and more particularly, to a sensor device for detecting analog Laplacian electroencephalogram (LEEG) signals and a method of detection thereof.

An electroencephalogram or EEG is a brain electrical activity recorded from the scalp. The main source of EEG is the synchronous activity of thousands of cortical neurons in the brain. The EEG has been applied to a Brain-Computer Interface (BCI) field for achieving a direct interface between human and machines through the EEG without language or body actions. For example, BCI systems can allow people with severe motor disabilities to use scalp-recorded EEG activity to control a device, such as computer curser or prosthesis.

Typically, EEG signals accompanying noise are captured and amplified by the EEG electrodes placed on the skull and an EEG instrument. Amplified signals and noise are digitized and transmitted to the PC for further signal processing. For example, an active electrode has been developed in U.S. Pat. No. 6,052,609 to Ripoche et al., the contents of which is incorporated by reference herein, in which an electrophysiological device was disclosed to provide a solution to the problem of noise interference with measurements which prevent their analysis. The electrophysiological device includes a set of electrodes for detecting electromagnetic signals, a plurality of corresponding operational amplifiers connected directly to the electrodes for signal amplification, and a memory means intended to store the amplified signals for the purpose of subsequent analysis. The device is not sensitive to detecting the EEG signal which can be discriminated and uniquely associated with members of a set of commands for controlling a device such as the prosthesis, a biofeedback signaling device, a vehicle or other machine when the surrounding noise is taken into account. This is particularly the case when the measurement is taken in an open environment without any shielding for the noise.

According to the sensor disclosed in U.S. Pat. No. 6,091,977 to Tarjan et al., the contents of which is incorporated by reference herein, a concentric-designed and locally sensitive sensor was mounted on the exterior of the body of a subject to detect electrical activity of skeletal muscles in the immediate area underlying the sensor to a depth of a few millimeters, but which was substantially insensitive to electrical activity occurring elsewhere. However, it is difficult to mount the sensor on the scalp of the subject in case the EEG measurement is taken from the head of the subject. Also, the signals acquired by the sensor cannot be used to directly control the prosthetic device without further offline analyses using other processing devices, such as an analog-to-digital (A/D) converter, digital signal processor (DSP), and computer system.

It is desirable to provide a sensor device that detects analog Laplacian electroencephalogram (LEEG) signals. It is also desirable to provide a method of detecting LEEG signals.

### BRIEF SUMMARY OF THE INVENTION

One aspect of the invention provides a sensor device for detecting Laplacian electroencephalogram (EEG) signals. The sensor device comprises a signal acquisition module having a central electrode and a plurality of radially-arranged electrodes placed on a scalp of a subject for acquiring brain signals. A signal processing module is coupled to the signal acquisition module to perform a Laplacian operation on the signals acquired by the signal acquisition module with equations including V0=Vc−Vm and

$$Vm = \frac{V 1 + V 2 + V 3 + ⋯ + Vn}{n}$$

so as to yield an analog Laplacian EEG signal. V0 is the signal output of the signal processing module, Vc is the signal acquired by the central electrode and Vm is an arithmetic mean of signals V1-Vn acquired by the radially-arranged electrodes.

Another aspect of the invention is to provide a compact and portable sensor device for detecting an EEG signal. The sensor device includes a signal acquisition module that acquires the EEG signals and a signal differentiating module coupled to the signal acquisition module to perform a Laplacian operation on the signals acquired by the signal acquisition module based on equations including V0=Vc−Vm and

$$Vm = \frac{V 1 + V 2 + V 3 + ⋯ + Vn}{n} .$$

V0 is the signal output of the signal processing module, Vc is the signal acquired by the central electrode and Vm is an arithmetic mean of signals V1-Vn acquired by the radially-arranged electrodes. The sensor device also includes a signal amplification module coupled to the signal differentiating module that amplifies a signal output of the signal differentiating module and a signal filtering module that filters an amplified signal output from the signal amplification module, so that noise signals are reduced to yield an analog Laplacian EEG signal.

A further aspect of the invention provides a compact and portable sensor device for real-time detecting EEG signal. The device comprises a central electrode coupled to a plurality of radially-arranged electrodes that acquire the EEG signal, a circuit coupled to the central electrode and the radially-arranged electrodes to subtract an average of the signals acquired by the radially-arranged electrodes from the signal acquired by the central electrode, an amplifier coupled to the circuit to amplify a signal output of the circuit and a filter coupled to amplifier in order to filter an amplified signal output of the amplifier, so that noise signals are reduced to yield an analog Laplacian EEG signal.

Another embodiment of the present invention comprises a method for detecting EEG which includes acquiring brain signals using a signal acquisition module placed on a scalp of a subject. A Laplacian operation is performed on the signals acquired by the signal acquisition module, with a signal processor based on equations including V0=Vc−Vm and

$$Vm = \frac{V 1 + V 2 + V 3 + ⋯ + Vn}{n}$$

so as to yield an analog Laplacian EEG signal. V0 is a signal output of the signal differentiating module, Vc is the signal acquired by the central electrode and Vm is an arithmetic mean of signals V1-Vn acquired by the radially-arranged electrodes.

Another embodiment of the present invention comprises a method for real-time detecting EEG which includes acquiring EEG signals using a signal acquisition module placed on a subject. A Laplacian operation is performed on the signals acquired by the signal acquisition module, with a signal differentiating module based on equations including V0=Vc−Vm and

$$Vm = \frac{V 1 + V 2 + V 3 + ⋯ + Vn}{n} .$$

V0 is a signal output of the signal differentiating module, Vc is the signal acquired by the central electrode and Vm is an arithmetic mean of signals V1-Vn acquired by the radially-arranged electrodes. The signal output is amplified using a signal amplification module connected to the signal differentiating module, and an amplified signal output of the signal amplification module is filtered using a signal filtering module, so that noise signals are reduced to yield an analog Laplacian EEG signal.

Another embodiment of the present invention comprises a method for detecting EEG signal which includes sequentially acquiring potentials using a central electrode and a plurality of radially-arranged electrodes. A difference between the potential measured at the central electrode and the average potential of the radially-arranged electrodes is computed using a circuit coupled to the central electrode and the radially-arranged electrodes to yield a laplaced signal. The laplaced signal is amplified using an amplifier coupled to the circuit, and an amplified laplaced signal is filtered using a filter, so that noise signals are reduced to yield an analog Laplacian EEG signal.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is an image illustrating a sensor device for detecting a Laplacian electroencephalogram (LEEG) signal according to an embodiment of the present invention;

FIG. 2 is a block diagram illustrating the sensor device for detecting the LEEG signal according to an embodiment of the present invention;

FIG. 3 is a circuit diagram illustrating layout of a signal processor according to an embodiment of the invention;

FIG. 4 is a flow chart illustrating the method of detecting the LEEG signal according to an embodiment of the present invention;

FIG. 5A is a signal distribution diagram showing the LEEG signal acquired by the device in an unshielded room according to one preferred embodiment of the invention;

FIG. 5B is a diagram showing a power spectrum produced after the LEEG signal shown in FIG. 5A is transformed according to one preferred embodiment of the invention;

FIG. 6A is a signal distribution diagram showing the EEG signal acquired by the conventional EEG instrument in a shielded room; and

FIG. 6B is a signal distribution diagram showing the LEEG signal after the EEG signal shown in FIG. 6A is processed offline in a personal computer (PC).

### DETAILED DESCRIPTION OF THE INVENTION

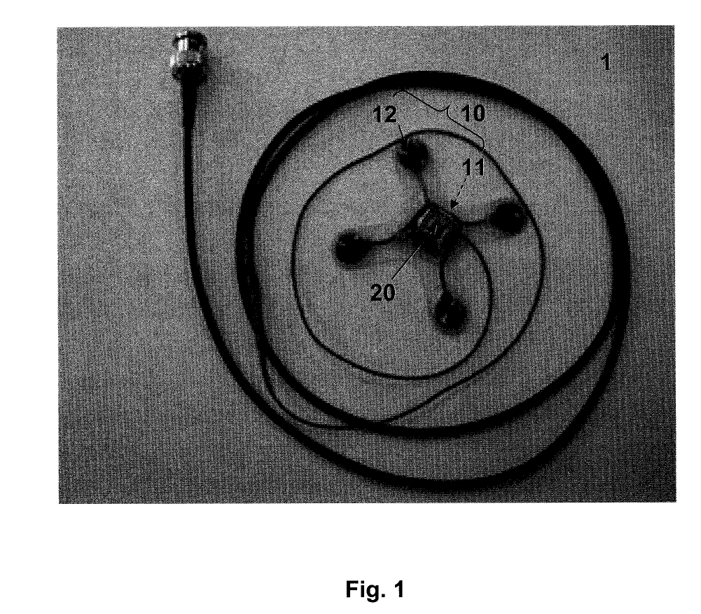

In accordance with one embodiment of the invention, the present invention provides a sensor device for detecting electroencephalogram (EEG) signal. Referring to FIG. 1, the sensor device 1 comprises a signal acquisition module 10 placed on a scalp of a subject to acquire brain signals and a signal processor 20 coupled or connected to the signal acquisition module to perform a Laplacian operation on the signals acquired by the signal acquisition module 10. The signal processor 20 may be coupled or connected adjacent to the signal acquisition module 10 or conveniently stacked on top of the signal acquisition module 10 to optimize space. The signal processor 20 may also be incorporated directly in the signal acquisition module 10 to form a signal detecting chip without involving any noticeable increase in the overall size of the device 1.

The signal acquisition module 10 comprises a central electrode 11, as indicated by a dotted line arrow for showing its preferred location underneath the signal processor 20, and a plurality of radially-arranged electrodes 12. In the preferred embodiment, the signal acquisition module 10 comprises five conductive electrodes 11 and 12 arranged in a cross-like configuration, with the central electrode 11 located at the center of the cross and two pairs of the electrodes 12 arranged radially away from the central electrode 11 as shown in FIG. 1. For example, the central electrode 11 is located at the center of the cross, and two pairs of the radially-arranged electrodes 12 are disposed about 3 centimeters (cm) from the center of the cross.

The radially-arranged electrodes 12 are affixed to peripheral ends of a cross-shaped silicon rubber structure. The radially-arranged electrodes 12 are coupled or connected to each other in series to the signal processor 20, with each of the radially-arranged electrodes 12 being disposed about 3 cm from the central electrode 11 specifically for detecting mu wave. Preferably, the signal acquisition module 10 comprises five gold electrodes 11 and 12 constructed to a cross-shaped silicon rubber structure with a battery-powered, low noise amplifier (LNA) incorporated as shown in FIG. 1. The signal acquisition module 10 may be optionally equipped with a reference electrode (not shown) for acquiring a reference signal or potential from the remote end, such as an ear, a forehead or a thigh of the subject.

The reference electrode may be dispensed with if, on the one hand, the number of electrodes is sufficient, and if, on the other hand, the signal acquisition module is capable of calculating a notional reference potential from the whole of the potentials delivered by the central electrode 11 and the radially-arranged electrodes 12.

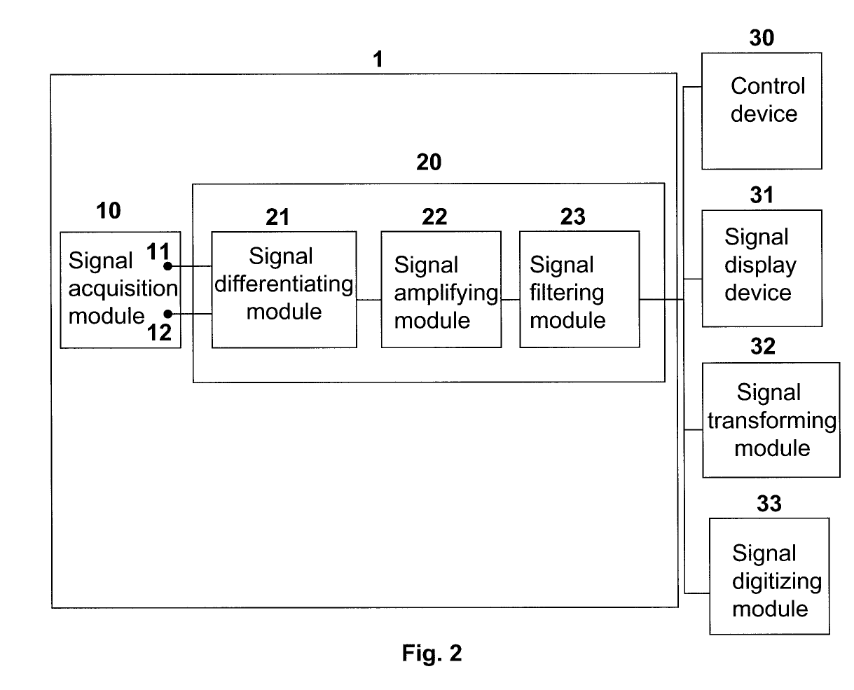

Referring to FIG. 2, the signal processor 20 further comprises a signal differentiating module 21, a signal amplification module 22 is coupled or connected to the signal differentiating module 21 and a signal filtering module 23 coupled or connected to the signal amplification module 22. The signal differentiating module 21, such as a circuit, performs a Laplacian operation on the signals acquired by the signal acquisition module 10 based on equations including V0=Vc−Vm and

$$Vm = \frac{V 1 + V 2 + V 3 + ⋯ + Vn}{n} ,$$

so as to yield an analog Laplacian EEG signal. V0 is a signal output of the signal differentiating module, Vc is the signal acquired by the central electrode, and Vm is an arithmetic mean of signals V1-Vn acquired by the radially-arranged electrodes 12. For example, the signal differentiating module 21 subtracts an arithmetic mean of signals acquired by the radially-arranged electrodes 12 from the signal acquired by the central electrode 11.

Preferably, the circuit subtracts an average of the signals acquired by the four radially-arranged electrodes 12 from the signal acquired by the central electrode 11 in a Laplacian operation to yield a laplaced signal. The Laplacian operation is performed on the signals acquired by the signal acquisition module 10 based on equations including V0=Vc−Vm and

$$Vm = \frac{V 1 + V 2 + V 3 + V 4}{n} .$$

V0 is a signal output of the signal differentiating module 20, Vc is the signal acquired by the central electrode 11 and Vm is an arithmetic mean of signals V1-Vn acquired by the four radially-arranged electrodes 12. It should be noted that the number of the radially-arranged electrodes 12 is not limited as described above as long as the electrodes 12 are arranged in a radial fashion to reduce or eliminate as much noise signal production as possible.

The signal output of the signal differentiating module 21 is then amplified using the signal amplification module 22, such as an amplifier coupled or connected to the signal differentiating module 21. A signal filtering module 23 is further coupled or connected to the signal amplification module 22 for filtering an amplified signal output of the signal amplification module 22, so as to yield an analog EEG signal of high signal-to-noise (S/N) ratio.

As shown in FIG. 2, the signal processor 20 may be directly coupled or connected to a control device 30 for controlling a prosthesis or wheel chair based on the analog EEG signal detected using the device 1 according to embodiments of the present invention. Other optional processing devices 31-33, including a signal display device 31, a signal transforming module 32, and a signal digitizing module 33 or the like, may be coupled or connected via a signal connector or wireless transmission to the signal processor 20 to further analyze the signals in different forms or to visually present the signals. The signal display device 31, such as a wave-reading device or programmed monitor, can display in real time at least some of the signals representing the evoked potentials emitted when the subject was cued to imagine grasping something. For example, the EEG signals acquired using the device 1 according to embodiments of the present invention may also be directly displayed on the oscilloscope through coaxial cable with a BNC signal connector.

The signal transforming module 32, for example a computer or other device for transforming the analog EEG signals into corresponding power spectrums, may be coupled or connected to the signal processor 20. The signal transforming module 32 preferably performs a Short Time Fourier Transformation (STFT) on the signal output Vout of the signal processor 20 to produce the power spectrum. The signal digitizing module 33 may include an Analog/Digital (A/D) converter that converts the analog signals into the digital signals and a Digital Signal Processor (DSP) that processes the digital signal in other subsequent analyses. While the signals output by the optional processing devices 31-33 may be implemented in actuating the control device 30, the analog LEEG signal detected can alternately be directly implemented in the control device 30 without any prior digitization. Therefore, embodiments of the present invention provide the device 1 for detecting the analog LEEG signal so as to real-time control the prosthetic device. In addition, additional noises created as a result of differential operation of digital signals are either minimized or eliminated to improve stability in the subsequent control tasks.

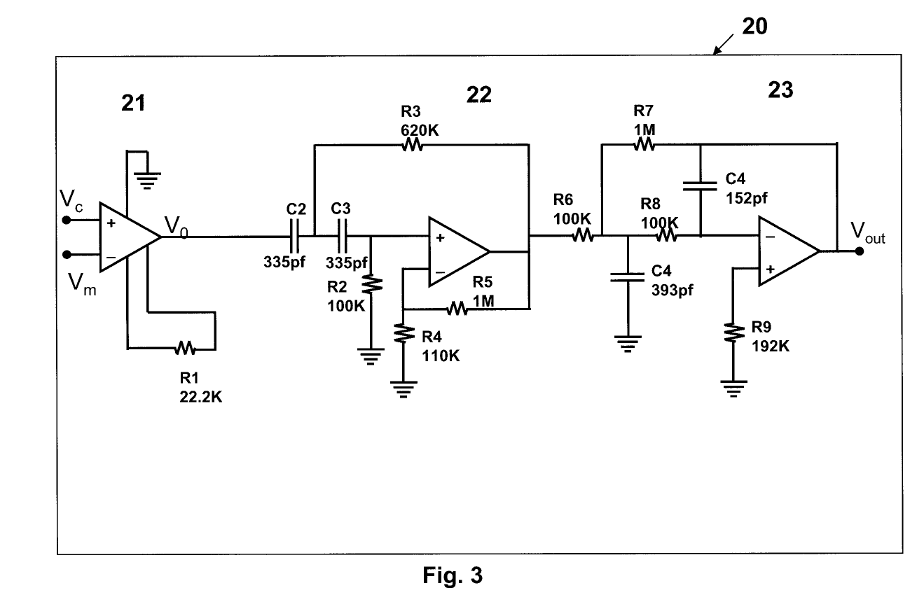

The operation of the signal processor 20 is now explained in more detail with reference to FIG. 3. As shown in the circuit diagram of the signal processor 20, the signals acquired by the signal acquisition module 10 are then subjected to a Laplacian operation conducted by the signal processor 20 which subtracts the average signal Vm based on signals V1, V2, V3, and V4 acquired by four radially-arranged electrodes 12 from the signal Vc acquired by the central electrode 11 according to the following equations to yield a signal output V0 of the signal differentiating module 21.

$$Vm = \frac{\begin{matrix}V 0 = Vc - Vm \\ V 1 + V 2 + V 3 + V 4\end{matrix}}{4}$$

Specifically, the signal differentiating module 21 may contain a differentiating amplifier with a gain of about 100 and a quasi high-pass filter with a frequency bandwidth of about 0.2 Hertz (Hz) constructed in such a way as illustrated in FIG. 3, so that after the signals Vc and Vm are both input to the signal differentiating module 21, the signal output V0 is produced. The signal input is then subject to further amplification with the signal amplification module 22 containing an amplifier having a gain of about 10 and a high-pass filter with the frequency bandwidth of about 0.2 Hz constructed in such a way as illustrated in FIG. 3. Next, an amplified signal output of the signal amplification module 22 passes through the signal filtering module 23 which contains an amplifier having a gain of about 10 and a low-pass filter with the frequency bandwidth of about 55 Hz constructed as shown in FIG. 3, so as to real-time yield an analog LEEG signal Vout which can be directly implemented to control the device, such as a prosthetic or wheel chair for anyone having limited limb movement.

The signal processor 21, preferably a low noise amplifier (LNA) with a lithium battery, is attached to the backside of the central electrode 11 using the adhesive such as 3140RTV, commercially available from Dow Corning. A number of solar panels (not shown) may also be coupled or connected to the LNA to provide alternative modes of electrical supply. The LNA has the following specification: a gain of about 10,000, bandpass filtering from about 2.5 Hz to 55 Hz, an input impedance of about 10 Giga-ohms (GO), and a common mode rejection ratio (CMRR) of about 110 decibels (dB).

By shortening the distance between the electrodes 12 and the low noise amplifier 21, the sensor device 1 of embodiments of the present invention can output a mu wave for severe motor disabilities. The sensor device 1 improves not only the signal to noise ratio of the mu wave but also the digital signal processing time. With the high input impedance and lightweight design, the sensor device 1 of embodiments of the present invention may be built within a helmet or a hat (not shown) to be worn by the subject.

According to another aspect of the invention, a method for detecting EEG signals is provided. The method comprises acquiring brain signals using a signal acquisition module placed on a scalp of a test subject. A Laplacian operation is performed on the signals acquired by the signal acquisition module using a signal processor based on equations including V0=Vc−Vm and

$$Vm = \frac{V 1 + V 2 + V 3 + ⋯ + Vn}{n} ,$$

so as to yield an analog Laplacian EEG signal. V0 is a signal output of the signal differentiating module, Vc is the signal acquired by the central electrode signals and Vm is an arithmetic mean of signals V1-Vn acquired by the radially-arranged electrodes 12. In the preferred embodiment, the signal processor 20 as constructed in FIG. 3 achieves a gain of about 10,000, bandpass filtering from about 2.5 Hz to 55 Hz, input impedance of about 10 GO, and a CMRR of about 110 dB.

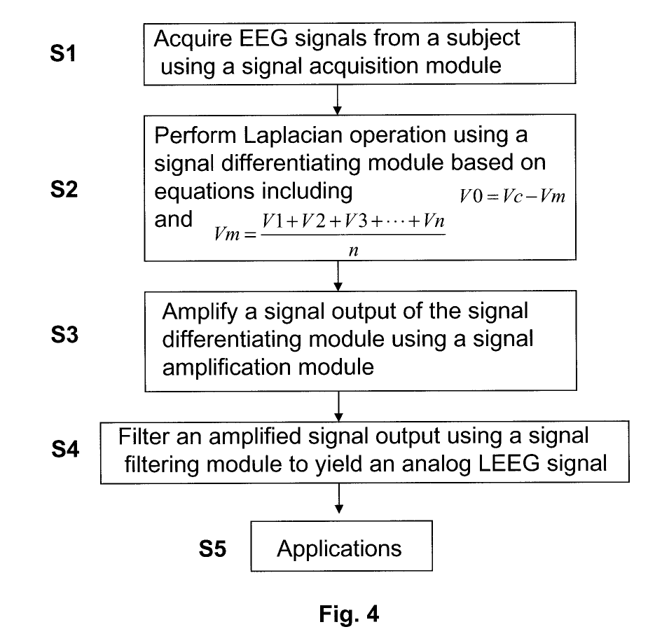

Referring to FIG. 4, the process for detecting an analog LEEG signal is described in a flow chart. In step S1, the EEG signal is acquired using a signal acquisition module 10 placed over a subject. The signal acquisition module 10, as described in FIGS. 1 and 2 above, comprises a central electrode 11 and a plurality of radially-arranged electrodes 12 arranged in a cross-like configuration. Preferably, the signal acquisition module 10 comprises a central electrode 11 disposed at the center of the cross and four radially-arranged electrodes 12, with each radially-arranged electrode 12 disposed about 3 cm away from the central electrode 11. The process then proceeds to step S2.

In step S2, a Laplacian operation is performed on the signals acquired by the signal acquisition module 10 using a signal differentiating module 21 based on equations including V0=Vc−Vm and

$$Vm = \frac{V 1 + V 2 + V 3 + ⋯ + Vn}{n} .$$

V0 is a signal output of the signal differentiating module, Vc is the signal acquired by the central electrode, and Vm is an arithmetic mean of signals acquired by the radially-arranged electrodes 12 having signals V1-Vn. Preferably, the signal differentiating module 21 subtracts an average of the signals V1-Vn acquired by the four radially-arranged electrodes 12 from the signal Vc acquired by the central electrode 11 based on the equations including V0=Vc−Vm and

$$Vm = \frac{V 1 + V 2 + V 3 + V 4}{n} .$$

The process then proceeds to step S3.

In step S3, a signal output of the signal differentiating module 21 is amplified using the signal amplification module 22 to produce an amplified signal output. The process then proceeds to step S4.

In step S4, the amplified signal output is filtered using a signal filtering module 23 with a preferable frequency bandwidth of about 0.2-55 Hz to yield an analog LEEG signal. The process then proceeds to applications in step S5.

The signal processing may optionally include one or more filtering operations, signal transformation and also digitalization after step S4. For example, the applications of step S5 may include performing STFT to transform the analog LEEG signal into a power spectrum, wherein the analog LEEG signal is implemented directly to the control device 30 for controlling the device such as the prosthesis and wheel chair. The applications of step S5 may include converting the analog LEEG signal into a digital signal, the analog LEEG signal being transformed by STFT using the signal transforming module 32 into a power spectrum to be more easily interpreted. The applications of step S5 may include controlling a device with the analog LEEG signal, the analog LEEG signal being converted into a digital signal for subsequent analysis. The analog LEEG signal may be converted using the signal digitizing module 33 which includes the A/D converter and DSP. The application of step S5 may include displaying the analog LEEG signal by using the signal display device 31. For example, the analog LEEG signal may be displayed with the oscilloscope or other wave-reading devices. The various applications of step S5 can occur alone or in combination. The various applications of step S5 can occur simultaneously since each step may proceed without requiring further processing of the analog LEEG signal.

Accordingly, embodiments of the present invention provide the method for detecting the analog LEEG signal which obtains a real-time analog LEEG signal to directly control the prosthesis, artificial limbs, wheelchair and devices that assist the disabled persons to carry out their daily activity. Since the Laplacian computation is performed before the signal amplification and filtering without prior digitization, the noise amplified as a result of A/D conversion and signal amplification is reduced or minimized. Therefore, the noise signals are reduced to yield the analog LEEG signal of high S/N ratio.

Embodiments of the present invention will now be described in further detail with reference to the following specific, non-limiting examples.

### EXAMPLE 1

### Detection of EEG signal

A 24 year old normal and healthy male subject without any prior history of neuromuscular disease was selected for participating in this study. According to an embodiment of the present invention, the sensor device 1 for detecting EEG signals was worn on the subject's head, with the central electrode 11 placed on the left Rolandic area, also known as sensorimotor area or so called C3 according to the international 10-20 system. The sensor device 1 comprises of five gold electrodes 12 constructed by embedding into a cross-shaped silicon rubber structure, with one surface of each electrode 12 exposed for picking up the signals. The signal electrodes 12 are placed in such a way that the central electrode 11 is placed at the center of the cross, and two pairs of the radially-arranged electrodes 12 are disposed at a distance, preferably about 3 cm away from the center of the cross. In this study, a common EEG paste was applied over a contact surface of each electrode 12 to achieve better conductivity between the scalp and electrodes.

During recording, the subject sat on a deck chair and was asked to remain motionless. Data was acquired by the device 1 for detecting EEG signal in the unshielded room as the subject was asked to imagine grasping something with his right hand during 5 to 15 seconds and 25 to 35 seconds. The subject was asked to keep his eyes closed at all time to reduce interference of the alpha rhythm. This might be achieved through the biofeedback training, whereby the subject was given an indication as to how well he/she was controlling a device (e.g. by looking at it). The subject then changed their EEG signal in response to this feedback. In this way, the subject learned to control the device through a learning process.

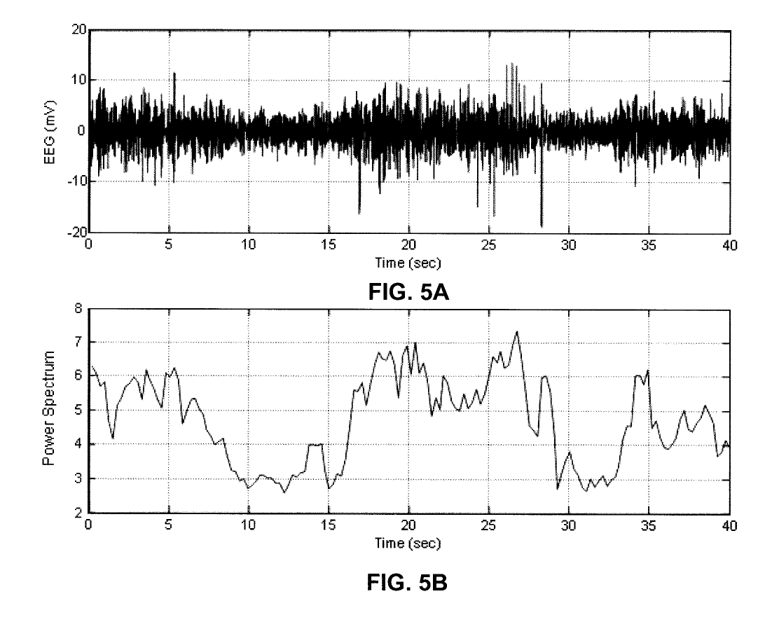

Referring to FIG. 5A, the analog LEEG signals of high S/N ratio were obtained, and the amplitude of the analog LEEG signals fell off in the duration of 5 to 15 seconds and 25 to 35 seconds. In this example, the analog LEEG signal detected using the sensor device and the method described above was a mu wave which is responsible for controlling the motor function of the subject. Therefore, the mu wave detected according to the invention would provide a control input command which could control a device/system quickly and accurately for the subjects with severe motor disabilities.

A power spectrum of mu wave might also be computed using the signal transforming module 32 according to the method described in FIG. 4 and shown in FIG. 5B. It was apparent that the power spectrum of mu wave was suppressed in the period of subject's imagination. The power spectrum was defined to extract mu waves from LEEG signals, and the power spectrum of mu waves would be adopted as a control input command. The computation process of power spectrum included the correction of STFT. Therefore, the power spectrum of mu wave yielded in real time by the sensor device of the invention was a control input command, which could control a device/system quickly and accurately for the patients with severe motor disabilities.

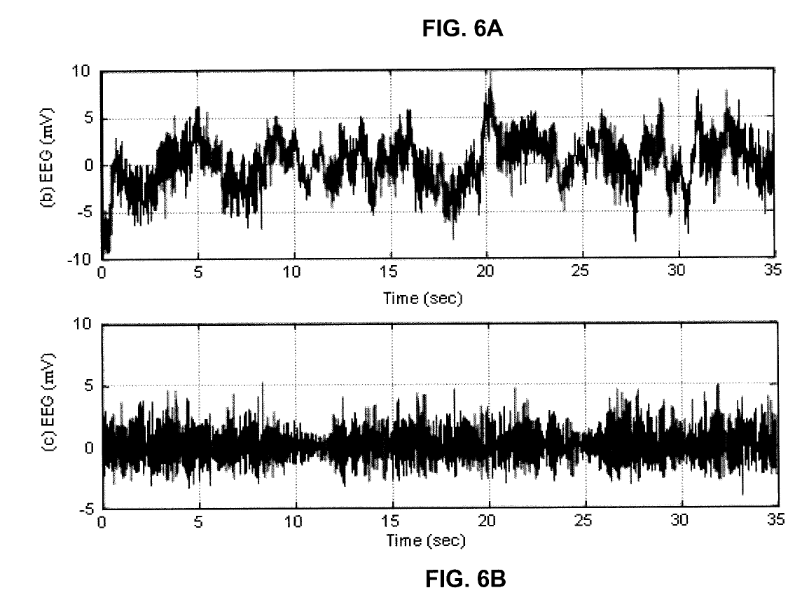

In a contrasting example, the experiment was also carried out in a shielded room of a hospital where investigators recorded EEG using the conventional sensor and the clinical EEG instrument (Oxford Instruments, amplification of 10,000) as the subject imagined his right hand grasping something at the periods of 8-12 seconds and 23-26 seconds. The EEG signal acquired by the sensor device was measured with the clinical EEG instrument as shown in FIG. 6A. However, it was difficult to inspect or interpret mu wave suppression from FIG. 6A since the signals measured by the EEG instrument were still accompanied with the high signal noise. Thus, the signals were subjected to a series of processing including digitizing, filtering with a 3-50 Hz bandpass filter, and laplacing the EEG signals offline in the PC to produce the digitalized LEEG versus time as depicted in FIG. 6B.

By examining the digitalized LEEG distribution as shown in FIG. 6B, it was found that the magnitude of digitalized LEEG was diminished or reduced at two time intervals between 8-12 seconds and 23-26 seconds, which was similar to the results delineated in FIG. 5A. Therefore, it was evident that the sensor device 1 could detect the analog LEEG signal in real time without resorting to the EEG instrument and subsequent offline analyses, while the analog LEEG signal detected might be directly implemented to control the prosthetic device.

Summarizing from the above, embodiments of the present invention provide a sensor device for detecting analog LEEG signals from the subject. The sensor device detects the analog LEEG signals in real time via the signal processor which performs the Laplacian operation on the signals acquired by the signal acquisition module. The laplaced signal is then amplified and filtered to yield the analog LEEG signal. Since the Laplacian computation is performed before the signal amplification and filtering, the error generated as a result of A/D conversion and signal amplification is minimized.

The power spectrum is defined to extract mu waves from LEEG signals and the power spectrum of mu waves will be adopted as a control input command. The sensor device for detecting the analog LEEG signal and the method thereof may also be employed for the application of other brain rhythms such as alpha, beta, and so on. Therefore, it would be understood by those having ordinary skill in the art that other EEG signals may also be detected by the sensor device and detecting method of the invention for implementing in other applications more efficiently than the conventional EEG instruments currently available.

From the foregoing, it can be seen that embodiments of the present invention include a sensor that detects LEEG signals and a method of detecting LEEG signals. It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A sensor device for detecting Laplacian electroencephalogram (LEEG) signals, comprises:
a signal acquisition module having a central electrode and a plurality of radially-arranged electrodes placed on a scalp of a subject to acquire brain signals;
a signal processing module coupled to the signal acquisition module, the signal processing module performing a Laplacian operation on the signals acquired by the signal acquisition module with equations including V0=Vc−Vm and
$$Vm = \frac{V 1 + V 2 + V 3 + … + Vn}{n} ,$$
wherein V0 is the signal output of the signal processing n module, Vc is the signal acquired by the central electrode and Vm is an arithmetic mean of signals V1-Vn acquired by the radially-arranged electrodes;
a signal amplification module coupled to the signal processing module, the signal amplification module amplifying the signal output; and
a signal filtering module coupled to the signal amplification module, the signal filtering module filtering an amplified signal output of the signal amplification module, so as to yield an analog LEEG signal.

2. The sensor device according to claim 1, wherein the signal filtering module comprises an approximately 2.5-55 Hertz (Hz) bandpass filter.

3. The sensor device according to claim 2, further comprising
an analog-to digital (A/D) converter coupled to the filter, the A/D converter converting the analog LEEG signal into a digital LEEG signal.

4. The sensor device according to claim 1, further comprising:
a signal translator coupled to the amplifier, the signal translator transforming the analog Laplacian EEG signal into a power spectrum using a Short Time Fourier Transform (STFT).

5. A method for detecting Laplacian electroencephalogram (LEEG) signals, the method comprising:
acquiring brain signals using a signal acquisition device placed on a scalp of a subject;
performing a Laplacian operation on the signals acquired by the signal acquisition module using a signal differentiating module based on equations including V0=Vc−Vm and
$$Vm = \frac{V 1 + V 2 + V 3 + … + Vn}{n}$$
wherein V0 is a signal output of the signal differentiating module, Vc is the signal acquired by the central electrode and Vm is an arithmetic mean of signals V1-Vn acquired by the radially-arranged electrodes;
amplifying the signal output using a signal amplification module coupled to the signal differentiating module; and
filtering an amplified signal output of the signal amplification module using a signal filtering module coupled to the signal amplification module, so as to yield an analog LEEG signal.

6. The method according to claim 5, wherein the signal filtering module comprises an approximately 2.5-55 Hz bandpass filter.

7. The method according to claim 6, further comprising:
converting the analog Laplacian EEG signal into a digital signal.

8. The method according to claim 5, further comprising:
transforming the analog Laplacian EEG signal into a power spectrum using a Short Time Fourier Transform (STFT).

9. The method according to claim 5, wherein the radially-arranged electrodes include four electrodes constructed in a cross configuration.
